SEQUENCE LISTING (30 sequences)

SEQ ID NO: 1 - DNA, 10 bp, Artificial Sequence
  Ribosome Binding Site Sequence
acaaaaagga

SEQ ID NO: 2 - DNA, 33 bp, Artificial Sequence
  Primer for PCR
cgagctacatatgcaatgattgacacgattccg

SEQ ID NO: 3 - DNA, 32 bp, Artificial Sequence
  Primer for PCR
cgcgcgaattctatttgttagtgaataaaagg

SEQ ID NO: 4 - DNA, 32 bp, Artificial Sequence
  Primer for PCR
cgcgcgcatgctatttgttagtgaataaaagg

SEQ ID NO: 5 - DNA, 35 bp, Artificial Sequence
  Primer for PCR
ccgctcgagcatatgctgtcgcaatgattgacacg

SEQ ID NO: 6 - DNA, 34 bp, Artificial Sequence
  Primer for PCR
gctattccatatgcagggttattgtctcatgagc

SEQ ID NO: 7 - DNA, 19 bp, Artificial Sequence
  Primer for PCR
tcggcacgtaagaggttcc

SEQ ID NO: 8 - DNA, 19 bp, Artificial Sequence
  Primer for PCR
cgggtcgaatttgctttcg

SEQ ID NO: 9 - DNA, 28 bp, Artificial Sequence
  Primer for PCR
ctagatctgacagtaagacgggtaagcc

SEQ ID NO: 10 - DNA, 29 bp, Artificial Sequence
  Primer for PCR
ctagatctcagggttattgtctcatgagc

SEQ ID NO: 11 - DNA, 45 bp, Artificial Sequence
  Primer for PCR
cctttggttaaaggctttaagatcttccagtggacaaactatgcc

SEQ ID NO: 12 - DNA, 45 bp, Artificial Sequence
  Primer for PCR
ggcatagtttgtccactggaagatcttaaagcctttaaccaaagg

SEQ ID NO: 13 - DNA, 53 bp, Artificial Sequence
  Primer for PCR
ccagtggagctccggagaaagtcttatgtcccccattgaaaaatccagcaaat

SEQ ID NO: 14 - DNA, 42 bp, Artificial Sequence
  Primer for PCR
ttaaacggtaccttacagctgatgataaccagaaaggaaacg

SEQ ID NO: 15 - DNA, 55 bp, Artificial Sequence
  Primer for PCR
ccagttggtacccggagaaagtcttatgcgtggagaattttatcagcagttaacc

SEQ ID NO: 16 - DNA, 41 bp, Artificial Sequence
  Primer for PCR
gacagtgtcgacttaatcccagctcagaataactttcccgg

SEQ ID NO: 17 - DNA, 56 bp, Artificial Sequence
  Primer for PCR
ttgagctcacaaaaaggataaaacaatgactataacaaaacagcttcatacaccgc

SEQ ID NO: 18 - DNA, 41 bp, Artificial Sequence
  Primer for PCR
aaaaggtaccttacagactagacaacgctacttcttcctcg

SEQ ID NO: 19 - DNA, 58 bp, Artificial Sequence
  Primer for PCR
tatcggatcccggagaaagtcttatgagtaagcgtaaagtcgccattatcggttctgg

SEQ ID NO: 20 - DNA, 32 bp, Artificial Sequence
  Primer for PCR
tatttctagatcatgccgcttctcctgccttg

SEQ ID NO: 21 - DNA, 55 bp, Artificial Sequence
  Primer for PCR
tatttctagacggagaaagtcttatgattgatttacgcagtgataccgttacccg

SEQ ID NO: 22 - DNA, 33 bp, Artificial Sequence
  Primer for PCR
cagtgtcgacttaacgcgccaggaatgcacgcc

SEQ ID NO: 23 - DNA, 50 bp, Artificial Sequence
  Primer for PCR
tatcgtcgaccggagaaagtcttatgttgatgttatttctcaccgtcgcc

SEQ ID NO: 24 - DNA, 38 bp, Artificial Sequence
  Primer for PCR
tagggtcgactcaccgcgaaataatcaaatgaatgcca

SEQ ID NO: 25 - DNA, 32 bp, Artificial Sequence
  Primer for PCR
ggaattccatatggctgttaaaaatcgtctac

SEQ ID NO: 26 - DNA, 29 bp, Artificial Sequence
  Primer for PCR
gctctagatcagaatctgattccgtgttc

SEQ ID NO: 27 - DNA, 27 bp, Artificial Sequence
  Primer for PCR
aaagagctcaccatcagttgcgttatg

SEQ ID NO: 28 - DNA, 26 bp, Artificial Sequence
  Primer for PCR
aaaggtaccgtcagactcctaacttc

SEQ ID NO: 29 - DNA, 27 bp, Artificial Sequence
  Primer for PCR
aaatctagaatgaaactctacaatctg

SEQ ID NO: 30 - DNA, 27 bp, Artificial Sequence
  Primer for PCR
aaagcatgcgtttaaccctagcgccac

(19) United States
(12) Patent Application Publication
(10) Pub. No.: US 2015/0111261 A1
(43) Pub. Date: Apr. 23, 2015

(54) L-THREONINE-PRODUCING ESCHERICHIA COLI AND METHOD FOR PRODUCING L-THREONINE USING THE SAME

(71) Applicant: Mitsui Chemicals, Inc., Tokyo (JP)

(72) Inventors: Dominik Lukas JURGEN-LOHMANN, Singapore (SG); Ryota Fujii, Singapore (SG); Tomonori Hidesaki, Singapore (SG)

(73) Assignee: Mitsui Chemicals, Inc., Tokyo (JP)

(21) Appl. No.: 14/059,178

(22) Filed: Oct. 21, 2013

(51) Int. Cl. C12P 13/08

(52) U.S. Cl. CPC C12P 13/08
USPC 435/115; 435/252.33

(57) ABSTRACT

An L-threonine-producing Escherichia coli including a pathway selected from the group consisting of a pathway that produces acetaldehyde from acetyl-CoA with acetaldehyde dehydrogenase, and a pathway that produces L-threonine from acetaldehyde and glycine with threonine aldolase, and a pathway that produces 2-amino-3-oxobutyrate from glycine and acetyl-CoA with glycine C-acetyltransferase, and a pathway that produces L-threonine from 2-amino-3-oxobutyrate with threonine dehydrogenase.

### TECHNICAL FIELD

The present invention relates to an L-threonine-producing Escherichia coli and a method for producing L-threonine, using the same.

### BACKGROUND ART

The use of L-threonine, particularly as an additive to animal feed, has a large economic significance. Production of L-threonine is carried out using microbial fermentation. Several examples for L-threonine overproducing microorganisms are known in the art, with those derived from Escherichia coli being the most prominent.

All L-threonine overproducers known in the art are deriving threonine from the common intermediate oxaloacetate, which is converted to L-aspartate which is converted to L-aspartyl phosphate, which is converted to L-aspartate semialdehyde, which is converted to L-homoserine, which is converted to L-homoserine phosphate, which is converted to L-threonine (V G Debabov, Advances in Biochemical Engineering/Biotechnology Volume 79, 2003, pp 113-136 (Non-patent Document 1)). Improvements to threonine overproduction described in the art are related to increasing the activities of enzymes related to the above mentioned pathway through overexpression and/or removal of feedback inhibition, reducing the extent of side reactions and increasing the export of L-threonine out of the bacterial cell. These improvements have been carried out through mutagenesis and screening (Non-patent Document 1) as well as through rational design (K H Lee et al., Molecular Systems Biology 3:149 (Non-patent Document 2)).

Several mechanisms for L-threonine degradation are known. For example, L-threonine can be further metabolized to L-isoleucine or to glycine and acetyl-CoA by threonine dehydrogenase and glycine C-acetyl transferase or threonine aldolase and acetaldehyde dehydrogenase. The “primary role” of these pathways was classified as “threonine catabolism” (J P Marcus & E E Dekker, J. Bacteriol., 1993, 175(20), 6505-6511 (Non-patent Document 3)).

### SUMMARY OF THE INVENTION

### Problems to be Solved by the Invention

Several descriptions of L-threonine overproducers are known in the art. They share a common L-threonine production pathway via oxaloacetate, L-aspartate, L-aspartyl phosphate, L-aspartate semialdehyde, L-homoserine and L-homoserine phosphate. Consumption of ATP and redox equivalents for these steps amount to 2 moles ATP and 2 moles NADPH per mole of L-threonine produced.

Non-patent Document 3 describes a different approach in which L-threonine is formed from glycine and acetyl-CoA by C-acetyl transferase and threonine dehydrogenase. However, Non-patent Document 3 also describes that the amount of L-threonine produced from glycine and acetyl-CoA by C-acetyl transferase and threonine dehydrogenase is insufficient.

An L-threonine overproducer that has a lower requirement for ATP and/or redox equivalents to form L-threonine from oxaloacetate is not known in the art, and utilization of threonine aldolase and acetaldehyde dehydrogenase for L-threonine overproduction is not known in the art, either.

The present invention was made under the above-described circumstances, and aims to provide an L-threonine-producing Escherichia coli capable of producing L-threonine with high efficiency in large amounts. The present invention also aims to provide a method for producing L-threonine with high efficiency using the L-threonine-producing Escherichia coli.

### Means for Solving the Problems

More specifically, aspects of the present invention include the following.

<1> An L-threonine-producing Escherichia coli including a pathway selected from the group consisting of a pathway that produces acetaldehyde from acetyl-CoA with acetaldehyde dehydrogenase, and a pathway that produces L-threonine from acetaldehyde and glycine with threonine aldolase, and a pathway that produces 2-amino-3-oxobutyrate from glycine and acetyl-CoA with glycine C-acetyltransferase, and a pathway that produces L-threonine from 2-amino-3-oxobutyrate with threonine dehydrogenase.
<2> The L-threonine-producing Escherichia coli according to <1>, further including a pathway that produces glycine from glyoxylate with glyoxylate transaminase.
<3> The L-threonine-producing Escherichia coli according to <1>, further including a pathway that produces glyoxylate and acetyl-CoA from malyl-CoA with malyl-CoA lyase.
<4> The L-threonine-producing Escherichia coli according to <1>, further including a pathway that produces malyl-CoA from malic acid and acetyl-CoA with malate thiokinase.
<5> A method of producing L-threonine, the method including:

culturing the L-threonine-producing Escherichia coli of any one of <1> to <4> in contact with a carbon source material; and

collecting L-threonine produced by the L-threonine-producing Escherichia coli.

### Advantageous Effects of Invention

According to the present invention, an L-threonine-producing Escherichia coli can be provided which is capable of producing L-threonine with high efficiency in large amounts. According to the present invention, a method for producing L-threonine with high efficiency by using the L-threonine-producing Escherichia coli can also be provided.

### BRIEF DESCRIPTION OF THE DRAWING

FIG. 1 is a diagram schematically illustrating the pathways related to production of L-threonine according to the invention.

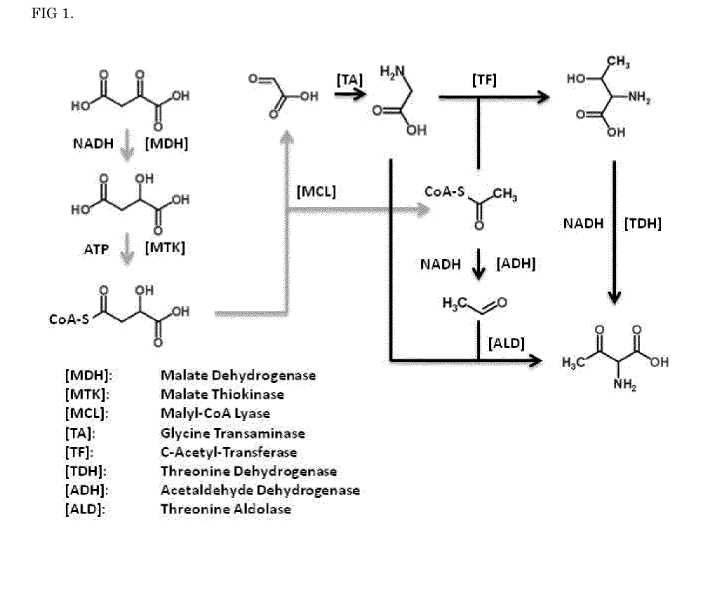

### BEST MODE FOR CARRYING OUT THE INVENTION

An L-threonine-producing Escherichia coli according to the invention is an Escherichia coli that possesses a pathway selected from the group consisting of a pathway that produces acetaldehyde from acetyl-CoA with acetaldehyde dehydrogenase, and a pathway that produces L-threonine from acetaldehyde and glycine with threonine aldolase, and a pathway that produces 2-amino-3-oxobutyrate from glycine and acetyl-CoA with glycine C-acetyltransferase, and a pathway that produces L-threonine from 2-amino-3-oxobutyrate with threonine dehydrogenase.

As described above, conventional approaches for L-threonine production have focused on an L-threonine production pathway via oxaloacetate, L-aspartate, L-aspartyl phosphate, L-aspartate semialdehyde, L-homoserine and L-homoserine phosphate since other pathways related to L-threonine have been considered to degrade L-threonine rather than generate L-threonine. However, the inventors of the present invention have found, for the first time, that L-threonine production, rather than L-threonine degradation, can be achieved by using acetaldehyde dehydrogenase and threonine aldolase. The present invention is described in detail below.

The term “step” as used in the present invention includes not only independent steps. Even when a step cannot be clearly distinguished from other steps, the step is included in the scope of this term as long as the expected purpose of the step can be achieved. Further, in the present specification, each numerical range represented using “to” means the range having the numerical values described before and after the “to” as the minimum value and the maximum value, respectively.

In the present invention, when the amount of each component in a composition is mentioned and the composition contains plural substances corresponding to the each component, the amount means the total amount of the plural substances contained in the composition unless otherwise specified.

The term “reduction” of an enzymatic and/or proteinaceous activity as used in the present invention means a state where the activity of an enzyme is significantly reduced by the gene recombination technology applied to the gene encoding the enzyme, compared to the state before carrying out the treatment.

The term “enhancement” of an activity as used in the present invention broadly means enhancement of various enzymatic and/or proteinaceous activities in Escherichia coli compared to the activities before the enhancement. The method of enhancement is not restricted as long as the activities of enzymes and/or functional proteins of Escherichia coli can be increased.

The term “impartation” of an activity as used in the present invention broadly means introduction of an enzyme and/or functional protein gene from the outside of a host bacterium into the inside thereof, and the scope thereof also encompasses enhancement of the activity of a promoter of an enzyme and/or functional protein gene retained in the genome of a host bacterium, and replacement of a promoter with another promoter to cause overexpression of an enzyme and/or functional protein gene.

The term “by a gene recombination technology” as used in the present invention includes any modification to a base sequence of a gene caused by insertion of another DNA to the base sequence of an inherent gene, or by replacement or deletion of a certain part of a gene, or any combination thereof. The modification may also be due to, for example, occurrence of a mutation.

The term “host” as used in the present invention means a microorganism which belongs to Escherichia coli.

The term “host” as used in the present invention further means a microorganism which belongs to Escherichia coli, and which inherently has a capacity to produce L-threonine from a carbon source material or can be made to have a capacity to produce L-threonine from a carbon source material by using a certain means.

The “host” in the present invention may have a pathway for production of a particular metabolite, which may be any metabolite generated in the metabolic pathways of microorganisms, such as an alcohol, an amino acid, an organic acid or a terpene. The microorganism may be a microorganism which has an inherent capacity to produce the particular metabolite, or a microorganism which does not have an inherent capacity to produce the particular metabolite and to which a capacity to produce the particular metabolite has been imparted by using a certain means.

The L-threonine-producing Escherichia coli according to the invention is an Escherichia coli having a pathway selected from the group consisting of a pathway that produces acetaldehyde from acetyl-CoA with acetaldehyde dehydrogenase, and a pathway that produces L-threonine from acetaldehyde and glycine with threonine aldolase, and a pathway that produces 2-amino-3-oxobutyrate from glycine and acetyl-CoA with glycine C-acetyltransferase, and a pathway that produces L-threonine from 2-amino-3-oxobutyrate with threonine dehydrogenase. Due to this configuration, the L-threonine-producing Escherichia coli according to the invention is capable of producing L-threonine with high efficiency in large amounts.

The L-threonine-producing Escherichia coli according to the invention preferably has an enhanced or imparted acetaldehyde dehydrogenase activity or an enhanced or imparted threonine aldolase activity or both. Such enhancement or impartation of the activity provides a higher efficiency in terms of L-threonine production.

Acetaldehyde dehydrogenase as referred to in the invention is the collective name of enzymes which are classified with the enzyme code 1.2.1.10 according to the report by the I.U.B. Enzyme Commission and catalyze reactions producing acetaldehyde from acetyl-CoA while converting NADH to NAD+.

Examples of acetaldehyde dehydrogenase include those derived from Escherichia such as Escherichia coli, those derived from Bacillus such as Bacillus megaterium, those derived from Serratia such as Serratia marcescens.

Threonine aldolase as referred to in the invention is the collective name of enzymes which are classified with the enzyme code 4.1.2.5 according to the report by the I.U.B. Enzyme Commission and catalyze reactions producing L-threonine from acetaldehyde and glycine.

Examples of threonine aldolase include those derived from Escherichia such as Escherichia coli, those derived from Pseudomonas such as Pseudomonas aeruginosa, those derived from Clostridium such as Clostridium acetobutylicum.

Further, the L-threonine-producing Escherichia coli according to the invention preferably has a pathway that produces 2-amino-3-oxobutyrate from glycine and acetyl-CoA with glycine C-acetyltransferase, and a pathway that produces L-threonine from 2-amino-3-oxobutyrate with threonine dehydrogenase.

Glycine C-acetyltransferase as referred to in the invention is the collective name of enzymes which are classified with the enzyme code 2.3.1.29 according to the report by the International Union of Biochemistry (I.U.B.) Enzyme Commission and catalyze reactions producing 2-amino-3-oxobutyrate from glycine and acetyl-CoA.

Examples of glycine C-acetyltransferase include those derived from Escherichia such as Escherichia coli, those derived from Bacillus such as Bacillus subtilis, those derived from Serratia such as Serratia marcescens.

Threonine dehydrogenase as referred to in the invention is the collective name of enzymes which are classified with the enzyme code 1.1.1.103 according to the report by the I.U.B. Enzyme Commission and catalyze reactions producing L-threonine from 2-amino-3-oxobutyrate while converting NADH to NAD+.

Examples of threonine dehydrogenase include those derived from Escherichia such as Escherichia coli, those derived from Bacillus such as Bacillus subtilis those derived from Streptomyces such as Streptomyces coelicolor.

The L-threonine-producing Escherichia coli preferably has an enhanced or imparted C-acetyl transferase activity or an enhanced or imparted threonine dehydrogenase activity or both. The enhanced or imparted activity or activities provide a higher efficiency in terms of L-threonine production.

Further, the L-threonine-producing Escherichia coli preferably possesses a pathway that produces glycine from glyoxylate with glyoxylate transaminase.

Glyoxylate transaminase as referred to in the invention is the collective name of enzymes which are classified with the respective enzyme codes 2.6.1.4, 2.6.1.44 or 2.6.1.45 according to the report by the I.U.B. Enzyme Commission and catalyze reactions producing glycine from glyoxylate using the respective amino donors L-glutamate, L-alanine or L-serine. Additionally, glyoxylate transaminase as referred to in the invention is the collective name of enzymes which, according to their classification, promiscuously catalyze reactions producing glycine from glyoxylate using an amino donor.

Examples of glyoxylate transaminase include those derived from Escherichia such as Escherichia coli, those derived from Bacillus such as Bacillus megaterium those derived from Hyphomicrobium such as Hyphomicrobium methylovorum.

The L-threonine-producing Escherichia coli preferably has an enhanced or imparted glyoxylate transaminase activity, which provides a higher efficiency in terms of L-threonine production.

The L-threonine-producing Escherichia coli preferably has at least one of (i) an enhanced or imparted acetaldehyde dehydrogenase activity, (ii) an enhanced or imparted threonine aldolase activity, or (iii) an enhanced or imparted glyoxylate transaminase activity, and more preferably has all of (i) an enhanced or imparted acetaldehyde dehydrogenase activity, (ii) an enhanced or imparted threonine aldolase activity, or (iii) an enhanced or imparted glyoxylate transaminase activity. For example, the L-threonine-producing Escherichia coli may have an enhanced or imparted AlaA activity, an enhanced or imparted MhpF activity and an enhanced or imparted LtaE activity. These configurations provide a higher efficiency in terms of L-threonine production, through establishment of a pathway that produces acetaldehyde from acetyl-CoA with acetaldehyde dehydrogenase, and a pathway that produces L-threonine from acetaldehyde and glycine with threonine aldolase.

Further, the L-threonine-producing Escherichia coli preferably has an enhanced or imparted system for glyoxylic acid production or an enhanced or imparted system for acetyl-CoA production or both. The enhancement or impartation of each system can be achieved by, for example, enhancement or impartation of the activities of one or more enzymes involved in the system (e.g., at least one of malate dehydrogenase, malate thiokinase or malyl-CoA lyase). These enhanced or imparted systems further improve the efficiency of L-threonine production by providing greater amounts of substances that can be converted to L-threonine by acetaldehyde dehydrogenase and threonine aldolase.

Further, the L-threonine-producing Escherichia coli according to the invention preferably has a pathway that produces glyoxylate and acetyl-CoA from malyl-CoA with malyl-CoA lyase.

Malyl-CoA lyase is an enzyme which is classified as enzyme code number 4.1.3.24 according to the report of the enzyme commission of International Union of Biochemistry (I.U.B.) and produces glyoxylate and acetyl-CoA from malyl-CoA. Examples of the enzyme include those derived from Methylobacterium such as Methylobacterium extorquens, Hyphomicrobium such as Hyphomicrobium methylovorum and Hyphomicrobium denitrificans, Chloroflexus such as Chloroflexus aurantiacus, Nitrosomonas such as Nitrosomonas europaea, and Methylococcus such as Methylococcus capsulatus.

In view of the efficiency of L-threonine production, especially preferred examples of the amino acid sequence of the malyl-CoA lyase include an amino acid sequence of a malyl-CoA lyase derived from Methylobacterium, an amino acid sequence of a malyl-CoA lyase derived from Hyphomicrobium, an amino acid sequence of a malyl-CoA lyase derived from Nitrosomonas, and an amino acid sequence of a malyl-CoA lyase derived from Methylococcus.

As the gene for the malyl-CoA lyase (mcl), a DNA having a base sequence of a gene encoding malyl-CoA lyase obtained from any of the above-mentioned source organisms or a synthetic DNA sequence synthesized based on a known base sequence of the gene encoding malyl-CoA lyase may be used. Preferred examples of the DNA include a DNA having a base sequences of a malyl-CoA lyase gene derived from Methylobacterium such as Methylobacterium extorquens, a DNA having a base sequence of a malyl-CoA lyase gene derived from Hyphomicrobium such as Hyphomicrobium methylovorum or Hyphomicrobium denitrificans, and a DNA having a base sequence of a malyl-CoA lyase gene derived from Chloroflexus such as Chloroflexus aurantiacus. In view of the efficiency of L-threonine production, especially preferred examples of the DNA include a DNA having a base sequence of a malyl-CoA lyase gene derived from Methylobacterium and a DNA having a base sequence of a malyl-CoA lyase gene derived from Hyphomicrobium.

The L-threonine-producing Escherichia coli preferably has an enhanced or imparted malyl-CoA lyase activity, which provides a higher efficiency in terms of L-threonine production.

The L-threonine-producing Escherichia coli according to the invention preferably has a pathway that produces malyl-CoA from malic acid and acetyl-CoA with malate thiokinase.

Malate thiokinase is classified as enzyme code number 6.2.1.9 according to the report of the enzyme commission of International Union of Biochemistry (I.U.B.), and is a general term for enzymes that bind malate to CoA to attain conversion to malyl-CoA. In this reaction, one molecule of ATP is consumed to produce one molecule each of ADP and phosphate. This enzyme is mainly found in assimilation pathways for C1 carbon sources such as methane (J. Bacteriol. 176(23), 7398-7404 (1994)) and 3-hydroxypropionate pathways (Arch. Microbiol., 151, 252-256 (1989)), and is characterized in that malyl-CoA lyase is present in the vicinity on the genome. Such an enzyme may be suitably used.

As the gene for the malate thiokinase (mtk), a DNA having a base sequence of a gene encoding malate thiokinase obtained from any of the above-mentioned source organisms or a synthetic DNA sequence synthesized based on a known base sequence of the gene encoding malate thiokinase may be used.

Preferred examples of the DNA include a DNA having a base sequence of a malate thiokinase gene derived from Methylobacterium such as Methylobacterium extorquens, a DNA having a base sequence of a malate thiokinase gene derived from Hyphomicrobium such as Hyphomicrobium methylovorum or Hyphomicrobium denitrificans, a DNA having a base sequence of a malate thiokinase gene derived from Rhizobium such as Rhizobium sp. NGR234, a DNA having a base sequence of a malate thiokinase gene derived from Granulibacter such as Granulibacter bethesdensis, a DNA having a base sequence of a malate thiokinase gene derived from Nitrosomonas such as Nitrosomonas europaea, a DNA having a base sequence of a malate thiokinase gene derived from Methylococcus such as Methylococcus capsulatus, and a DNA having a base sequence of a malate thiokinase gene derived from Gammaproteobacteria.

The L-threonine-producing Escherichia coli preferably has an enhanced or imparted malate thiokinase activity, which provides a higher efficiency in terms of L-threonine production.

The L-threonine-producing Escherichia coli according to the invention may further include one or more other enzymes from the viewpoint of efficient mass production of L-threonine. Examples thereof include those described below.

Malate dehydrogenase as referred to in the invention is the collective name of enzymes which are classified with the enzyme code 1.1.1.37 according to the report by the I.U.B. Enzyme Commission and catalyze reactions producing malate from oxaloacetate while converting NADH to NAD+.

Examples of malate dehydrogenase include those derived from Escherichia such as Escherichia coli, those derived from Corynebacterium such as Corynebacterium glutamicum, those derived from Bacillus such as Bacillus subtilis.

Threonine efflux protein as referred to in the invention is the collective name of functional proteins that facilitate selective transport of threonine from inside the cell through outside of the cell.

Examples of threonine efflux protein include those derived from Escherichia such as Escherichia coli, those derived from Serratia such as Serratia marcescens, those derived from Pantoea such as Pantoea ananatis.

The activity of each of these enzymes and/or functional proteins in the invention may be introduced from the outside of the host bacterium into the inside of the host bacterium, or alternatively, the activity of each of these enzymes in the invention may be realized by overexpression of the enzyme and/or functional protein genes by enhancement of activity of a promoter(s) of the enzyme and/or functional protein genes retained in the genome of the host bacterium or replacement of the promoter(s) with another promoter(s) to cause overexpression of the enzyme and/or functional protein gene.

Introduction of the enzyme and/or functional protein activities may be carried out, for example, by introduction of the genes encoding those kinds of enzymes and/or functional protein (any one or more of acetaldehyde dehydrogenase, threonine aldolase, glyoxylate transaminase, malyl-CoA lyase, malate thiokinase, malate dehydrogenase, glycine C-acetyltransferase, threonine dehydrogenase, or threonine efflux protein) from the outside of the host bacterium into the inside of the host bacterium using gene recombination technology. In this case, the origin of the introduced enzyme and/or functional protein genes may be either the same as or different from the species of the host cell. Preparation of the genomic DNA, cleavage and ligation of a DNA, transformation, PCR (Polymerase Chain Reaction), design and synthesis of oligonucleotides used as primers, and the like may be carried out by conventional methods well-known by persons skilled in the art. These methods are described in, for example, Sambrook, J., et al., “Molecular Cloning A Laboratory Manual, Second Edition”, Cold Spring Harbor Laboratory Press (1989).

Any promoter may be used as the promoter used for enhancement of the promoter activity or overexpression of the enzyme and/or functional protein gene as long as it is functional in a host such as Escherichia coli.

For example, promoters derived from E. coli or any other gram negative or gram positive bacteria may be used. Inducible strong promoters, more preferably strong constitutive promoters, may be used. For example, the lac, tac or gap promoter may be used.

These promoters may be introduced into the host cell according to a conventional method. Expression of the target enzyme and/or functional protein gene using a promoter may be carried out, for example, as follows. Specifically, the target enzyme and/or functional protein gene and the promotor may be ligated to the same vector, and the vector harbouring the target enzyme and/or functional protein gene and the promotor may be introduced into the host cell together with the enzyme and/or functional protein gene.

A ribosome binding site preceding the coding sequence of the gene to be introduced may be added. The ribosome binding site sequence can be any sequence that allows binding of the ribosome complex for subsequent translation in E. coli. The sequence preferably has some similarity to the ribosome binding site consensus sequence well known to a person skilled in the art, and the sequence is more preferably ACAAAAAGGA (SEQ ID NO: 1).

The introduction of genes into the host cell may be carried out using a plasmid system or genomic integration. Plasmid vectors can be any vector that can be maintained in an E. coli cell, and examples thereof include pBR322, derivatives thereof and others. The techniques employed for genome introduction or plasmid transformation are well known to any person skilled in the art. Any combination of plasmid transformation and/or genome introduction may be suitable in certain embodiments of the invention.

A method of producing L-threonine according to the invention includes:

culturing the L-threonine-producing Escherichia coli described above in contact with a carbon source material; and

collecting L-threonine produced by the L-threonine-producing Escherichia coli.

In the present invention, the method of producing L-threonine includes culturing the L-threonine-producing Escherichia coli in contact with a carbon source material. In preferable embodiments, the Escherichia coli is cultured in an appropriate medium while supplying a gas to the culturing mixture and collecting the L-threonine produced by the culturing mixture.

The method for production of L-threonine according to the present invention includes producing L-threonine from a suitable carbon source using the L-threonine producing Escherichia coli of the invention. That is, the method for producing L-threonine includes culturing the L-threonine producing Escherichia coli in a mixture containing the L-threonine producing Escherichia coli and a suitable carbon source, to allow the L-threonine producing Escherichia coli to assimilate the carbon source (hereinafter referred to as “culturing step”); and recovering the L-threonine obtained by the contact (hereinafter referred to as “recovering step”).

According to the method for producing L-threonine, since the L-threonine-producing Escherichia coli is brought into contact with a carbon source material when Escherichia coli is cultured, the carbon source material is assimilated by L-threonine-producing Escherichia coli, and the product of interest can be efficiently produced.

The carbon source is not particularly restricted as long as it can be metabolized by the L-threonine-producing bacterium of the invention. The carbon source may refer to any carbon source, examples of which include sucrose, glucose, arabinose and other saccharides as well as glycerin, amino acids and fatty acids.

In the culturing step, the L-threonine-producing Escherichia coli is generally brought into contact with a plant-derived material by culturing the L-threonine-producing Escherichia coli in a medium containing the plant-derived material.

The mixture in the production method for L-threonine may contain mainly a basal medium generally used for culturing Escherichia coli, and any medium may be used as long as it is a medium normally used depending on the type of the L-threonine producing Escherichia coli. Such a basal medium is not particularly limited as long as it is a medium containing a carbon source, nitrogen source, inorganic ions and, as required, other minor components.

In addition, the culture medium may contain other additive components which are usually added to media for Escherichia coli, such as antibiotics, at concentrations at which they are usually used. An antifoaming agent is preferably added to the culture medium at an appropriate amount for suppressing foaming.

There is no particular limitation to the culture condition employed for culturing according to the invention. In embodiments, culturing may be carried out under aerobic condition with appropriately controlling pH and temperature so that the pH becomes in the range of 4 to 9, preferably in the range of 6 to 8, and the temperature becomes in the range of 20 to 50 degrees Celsius, preferably in the range of 25 to 42 degrees Celsius.

Production of L-threonine may be accomplished by introducing activities related to L-threonine production, namely a acetaldehyde dehydrogenase and threonine aldolase and optionally one or more of the following activities, (1) a glyoxylate transaminase, (2) a malyl-CoA lyase, (3) a malate thiokinase, using the inherent activity of malate dehydrogenase, and (4) a glycine C-acetyltransferase and threonine dehydrogenase, thus enabling an Escherichia coli to produce L-threonine from oxaloacetate, which is naturally derived from a carbon source, with less ATP and/or redox equivalent requirement than L-threonine producing Escherichia coli known in the art. Particularly, the L-threonine producing Escherichia coli is capable of producing 1 mole of L-threonine from 1 mole of oxaloacetate, which is naturally derived from a carbon source, with the requirements of 1 mole ATP and 2 mole NADH. It is known in the art that providing NADH is energetically favourable compared to providing NADPH and can be considered a reduction in redox equivalent requirement. Thus, the advantage of less ATP and redox equivalent requirement can be envisioned by a person skilled in the art to be an improvement in theoretical yield of L-threonine.

The culturing step may be continued from the beginning of the culture until the carbon source material in the mixture has been consumed, or until the activity of the L-threonine-producing Escherichia coli disappears. The time period of the culturing step varies depending on the number and the activity of the L-threonine-producing Escherichia coli in the mixture, and on the amount of the carbon source material. In general, the time period may be not less than 1 hour, preferably not less than 4 hours. By additionally feeding the carbon source material and/or the L-threonine-producing Escherichia coli, the culture period can be extended without limitation, but, in view of the treatment efficiency, the culture period may be generally not more than 5 days, preferably not more than 72 hours. In terms of other conditions, those used in normal culture may be applied as they are.

The method for recovering L-threonine accumulated in the culture liquid is not restricted, and examples of the method which may be employed include a method wherein Escherichia coli cells are removed from the culture liquid by centrifugation or the like, followed by separating the product of interest by a normal separation method such as crystallization or chromatographic separation or membrane separation under conditions dependent on the type of the product of interest.

The method of the present invention for producing L-threonine may include, before the culturing step, a pre-culturing step for appropriately adjusting the number of cells and/or the activity state of the L-threonine-producing Escherichia coli used. The pre-culturing step is not restricted as long as it is a culture under conditions normally employed depending on the type of the L-threonine-producing Escherichia coli.

### EXAMPLES

The present invention is described in detail by way of Examples below. However, the present invention is not restricted by the Examples.

### Example 1

### Preparation of Plasmids pBRgapP and pMWGKC

In order to obtain the GAPDH promoter, PCR amplification was carried out using genomic DNA of the Escherichia coli MG1655 strain as a template, and primers CGAGCTACATATGCAATGATTGACACGATTCCG (SEQ ID NO: 2) and CGCGCGAATTCTATTTGTTAGTGAATAAAAGG (SEQ ID NO: 3). The amplified DNA fragment was digested with the restriction enzymes NdeI and EcoRI, to obtain a DNA fragment of about 110 bp corresponding to the GAPDH promoter. The obtained DNA fragment was mixed with the fragment obtained by digesting the plasmid pTH18cs1 (GenBank accession number AB019610) with the restriction enzymes NdeI and EcoRI, and the fragments were ligated using ligase. Thereafter, competent cells of the Escherichia coli DH5 alpha strain (Toyobo Co., Ltd., DNA-903) were transformed with the resulting ligation product, and transformants growing at 30° C. on an LB agar plate supplemented with 10 micro g/mL chloramphenicol were obtained. The obtained colonies were cultured in LB broth supplemented with 10 micro g/mL chloramphenicol at 30° C. overnight, and the plasmid pTH18gapP was recovered from the obtained bacterial cells. Subsequently, pTH18gapP was treated with restriction enzymes NdeI and HindIII and a ca. 160 bp fragment comprising the GAPDH promoter and a multi cloning site was recovered. This fragment was ligated with pBR322 (GenBank accession number J01749) which had been digested with NdeI and HindIII and this mixture was used to transform competent cells of the Escherichia coli DH5 alpha strain (Toyobo Co., Ltd., DNA-903). Transformants growing on an LB agar plate supplemented with 100 micro g/mL ampicillin were obtained. The obtained colonies were cultured in LB broth supplemented with 100 micro g/mL ampicillin overnight, and the plasmid pBRgapP was recovered from the obtained bacterial cells.

Similarly, in order to obtain the GAPDH promoter, PCR amplification was carried out using genomic DNA of the Escherichia coli MG1655 strain as a template, and primers CGAGCTACATATGCAATGATTGACACGATTCCG (SEQ ID NO: 2) and CGCGCGCATGCTATTTGTTAGTGAATAAAAGG (SEQ ID NO: 4). The amplified DNA fragment was digested with the restriction enzymes NdeI and SphI, to obtain a DNA fragment of about 110 bp corresponding to the GAPDH promoter. The obtained DNA fragment was mixed with the fragment obtained by digesting the plasmid pBR322 (GenBank accession number J01749) with the restriction enzymes NdeI and SphI, and the fragments were ligated together using ligase. Thereafter, competent cells of the Escherichia coli DH5α strain (Toyobo Co., Ltd., DNA-903) were transformed with the resulting ligation product, and transformants growing on an LB agar plate supplemented with 50 μg/mL ampicillin were obtained. The obtained colonies were cultured in LB broth supplemented with 50 μg/mL ampicillin at 37° C. overnight, and the plasmid pBRgapP_{—}2 was recovered from the obtained bacterial cells.

Subsequently, PCR amplification was carried out using pBRgapP_{—}2 as a template, and primers CCGCTCGAGCATATGCTGTCGCAATGATTGACACG (SEQ ID NO: 5) and GCTATTCCATATGCAGGGTTATTGTCTCATGAGC (SEQ ID NO: 6). The amplified DNA fragment was phosphorylated using T4 Polynucleotide Kinase (Takara), to obtain a DNA fragment containing a GAPDH promoter. Further, the plasmid pMW119 (GenBank accession number AB005476) was treated with the restriction enzymes AatII and NdeI, and the ends of the digested DNA fragment were blunt-ended with KOD plus DNA polymerase (Takara), to obtain a DNA fragment having the origin of replication of pMW119. The DNA fragments containing a GAPDH promoter and the origin of replication of pMW119 were ligated together using ligase. Thereafter, competent cells of the Escherichia coli DH5 alpha strain were transformed with the resulting ligation product, and transformants growing on an LB agar plate supplemented with 50 micro g/mL ampicillin were obtained. An obtained colony was cultured in LB broth supplemented with 50 micro g/mL ampicillin at 37° C. overnight, and the plasmid pMWG was recovered from the obtained bacterial cells.

In order to obtain a chloramphenicol resistance gene, PCR amplification was carried out using pTH18cs1 (GenBank accession No. AB019610) as a template, and primers TCGGCACGTAAGAGGTTCC (SEQ ID NO: 7) and CGGGTCGAATTTGCTTTCG (SEQ ID NO: 8), and the obtained DNA fragment was phosphorylated using T4 Polynucleotide Kinase (Takara), to obtain a DNA fragment containing a chloramphenicol resistance gene. Subsequently, PCR amplification was carried out using pMWG as a template, and primers CTAGATCTGACAGTAAGACGGGTAAGCC (SEQ ID NO: 9) and CTAGATCTCAGGGTTATTGTCTCATGAGC (SEQ ID NO: 10). The resulting fragment was mixed with the DNA fragment containing a chloramphenicol resistance gene and ligated together using ligase. Competent cells of the Escherichia coli DH5 alpha strain were transformed with the resulting ligation product, to obtain transformants growing on an LB agar plate supplemented with 25 micro g/mL chloramphenicol. An obtained colony was cultured in LB broth supplemented with 25 micro g/mL chloramphenicol at 37° C. overnight, and the obtained plasmid was designated pMWGC.

PCR amplification was carried out using the pMWGC gene as a template, and primers CCTTTGGTTAAAGGCTTTAAGATCTTCCAGTGGACAAACTATGCC (SEQ ID NO: 11) and GGCATAGTTTGTCCACTGGAAGATCTTAAAGCCTTTAACCAAAGG (SEQ ID NO: 12), followed by performing transformation of competent cells of the Escherichia coli DH5 alpha strain. Thereafter, transformants growing on an LB agar plate supplemented with 25 micro g/mL chloramphenicol were obtained. An obtained colony was cultured in LB broth supplemented with 25 micro g/mL chloramphenicol at 37° C. overnight, and the plasmid pMWGKC was recovered from the obtained bacterial cells.

### Example 2

### Preparation of Plasmid pBRgapP_alaA_kbl_tdh and pBRgapP_BMD_{—}3035_kbl_tdh

A ca. 1200 bp DNA fragment containing the ORF for alanine transaminase alaA was amplified from Escherichia coli MG1655 genomic DNA by PCR using oligonucleotides CCAGTGGAGCTCCGGAGAAAGTCTTATGTCCCCCATTGAAAAATCCAGCAAAT (SEQ ID NO: 13) and TTAAACGGTACCTTACAGCTGATGATAACCAGAAAGGAAACG (SEQ ID NO: 14). Subsequently, a ca. 2200 bp DNA fragment comprising the ORFs for glycine C-acetyltransferase kbl and threonine dehydrogenase tdh were amplified from Escherichia coli MG1655 genomic DNA by PCR using oligonucleotides CCAGTTGGTACCCGGAGAAAGTCTTATGCGTGGAGAATTTTATCAGCAGTTAACC (SEQ ID NO: 15) and GACAGTGTCGACTTAATCCCAGCTCAGAATAACTTTCCCGG (SEQ ID NO: 16). The obtained fragments were treated with KpnI, mixed and treated with ligase. Subsequently, a ca. 3400 bp DNA fragment was obtained from the ligation mix by PCR using oligonuceotides CCAGTGGAGCTCCGGAGAAAGTCTTATGTCCCCCATTGAAAAATCCAGCAAAT (SEQ ID NO: 13) and GACAGTGTCGACTTAATCCCAGCTCAGAATAACTTTCCCGG (SEQ ID NO: 16). This fragment was treated with SacI and SalI, mixed with pBRgapP from Example 1, which had been treated with SacI and SalI, and treated with ligase. The thus obtained plasmid was designated pBRgapP_alaA_kbl_tdh.

A ca. 1250 bp DNA fragment containing the ORF for class V aminotransferase BMD_{—}3035 from Bacillus megaterium DSM 319 was amplified using oligonucleotides TTGAGCTCACAAAAAGGATAAAACAATGACTATAACAAAACAGCTTCATACACCGC (SEQ ID NO: 17) and AAAAGGTACCTTACAGACTAGACAACGCTACTTCTTCCTCG (SEQ ID NO: 18) and genomic DNA from Bacillus megaterium DSM 319 as a template. The obtained fragment was digested with SacI and KpnI and ligated with aforementioned pBRgapP_alaA_kbl_tdh that had been digested with SacI and KpnI. This ligation mixture was used to transform Escherichia coli NEB5 alpha. The resulting plasmid was designated pBRgapP_BMD_{—}3035_kbl_tdh.

### Example 3

### Preparation of Plasmid pBRgapP_alaA_mhpF_ltaE

A ca. 1200 bp DNA fragment containing the ORF for alanine transaminase alaA was amplified from Escherichia coli MG1655 genomic DNA by PCR using oligonucleotides CCAGTGGAGCTCCGGAGAAAGTCTTATGTCCCCCATTGAAAAATCCAGCAAAT (SEQ ID NO: 13) and TTAAACGGTACCTTACAGCTGATGATAACCAGAAAGGAAACG (SEQ ID NO: 14). This fragment was treated with SacI and KpnI and ligated with pBRgapP as described in Example 1 which had been treated with SacI and KpnI. This ligation mixture was used to transform Escherichia coli NEB5 alpha. The resulting plasmid was designated pBRgapP_alaA.

A ca. 950 bp DNA fragment containing the ORF for acetaldehyde dehydrogenase mhpF was amplified from Escherichia coli MG1655 genomic DNA by PCR using oligonucleotides TATCGGATCCCGGAGAAAGTCTTATGAGTAAGCGTAAAGTCGCCATTATCGGTTCTG G (SEQ ID NO: 19) and TATTTCTAGATCATGCCGCTTCTCCTGCCTTG (SEQ ID NO: 20). Subsequently, a ca. 1000 bp DNA fragment containing the ORF for threonine aldolase ltaE was amplified from Escherichia coli MG1655 genomic DNA by PCR using oligonucleotides TATTTCTAGACGGAGAAAGTCTTATGATTGATTTACGCAGTGATACCGTTACCCG (SEQ ID NO: 21) and CAGTGTCGACTTAACGCGCCAGGAATGCACGCC (SEQ ID NO: 22). These fragments were treated with XbaI and ligase and a ca. 1950 bp DNA fragment was obtained by PCR from the ligation mixture using oligonucleotides TATCGGATCCCGGAGAAAGTCTTATGAGTAAGCGTAAAGTCGCCATTATCGGTTCTG G (SEQ ID NO: 19) and CAGTGTCGACTTAACGCGCCAGGAATGCACGCC (SEQ ID NO: 22). Plasmid pBRgapP_alaA was treated with BamHI and SalI and ligated with the aforementioned ca. 1950 bp fragment which had been treated with BamHI and SalI. This ligation mixture was used to transform Escherichia coli NEB5 alpha. The resulting plasmid was designated pBRgapP_alaA_mhpF_ltaE.

### Example 4

### Preparation of Plasmids pBRgapP_alaA_kbl_tdh_rhtC, pBRgapP_alaA_mhpF_ltaE_rhtC and pBRgapP_rhtC

A ca. 620 bp DNA fragment containing the ORF for threonine efflux protein rhtC was amplified from Escherichia coli MG1655 genomic DNA by PCR using oligonucleotides TATCGTCGACCGGAGAAAGTCTTATGTTGATGTTATTTCTCACCGTCGCC (SEQ ID NO: 23) and TAGGGTCGACTCACCGCGAAATAATCAAATGAATGCCA (SEQ ID NO: 24). Subsequently, this DNA fragment was treated with SalI and ligated with pBRgapP_alaA_kbl_tdh which had been treated with SalI. The correct orientation of rhtC in the obtained plasmid was verified and the plasmid was designated pBRgapP_alaA_kbl_tdh_rhtC.

Similarly, the ca. 620 bp DNA fragment containing the ORF for threonine efflux protein rhtC was treated with SalI and ligated with pBRgapP_alaA_mhpF_ltaE which had been treated with SalI. The correct orientation of rhtC was verified and the obtained plasmid was designated pBRgapP_alaA_mhpF_ltaE_rhtC.

Lastly, the aforementioned ca. 620 bp DNA fragment was treated with SalI and ligated with pBRgapP which had been treated with SalI. The correct orientation of rhtC was verified and the obtained plasmid was designated pBRgapP_rhtC.

### Example 5

### Preparation of Plasmid pMWGKC_mcl(Mc)_mtk(Mc)

Genomic DNA of Methylococcus capsulatus ATCC 33009D-5 was purchased from ATCC. PCR was carried out using the chromosomal DNA of Methylococcus capsulatus as a template, and primers GGAATTCCATATGGCTGTTAAAAATCGTCTAC (SEQ ID NO: 25) and GCTCTAGATCAGAATCTGATTCCGTGTTC (SEQ ID NO: 26) to obtain a mcl-mtk (malyl-CoA lyase—malate thiokinase) fragment of Methylococcus. The fragment was digested with NdeI and XbaI and ligated to the DNA fragment obtained by digesting the plasmid pMWGKC prepared in Example 1 with NdeI and XbaI. The thus obtained plasmid was designated as pMWGKC_mcl(Mc)_mtk(Mc).

### Example 6

### Preparation of W3110_thrB⁻

A ca. 700 bp fragment corresponding to the upstream region of thrB was amplified from W3110 genomic DNA by PCR using oligonucleotides AAAGAGCTCACCATCAGTTGCGTTATG (SEQ ID NO: 27) and AAAGGTACCGTCAGACTCCTAACTTC (SEQ ID NO: 28). The fragment was treated with SacI and KpnI and ligated with pTH18cs1 which had been treated with SacI and KpnI. Subsequently, E. coli DH5 alpha was transformed with the aforementioned ligation mixture and a clone carrying pTH18cs1 including the ca. 700 bp fragment was obtained after selection on an LB chloramphenicol plate (10 micro g/mL) at 30° C. Subsequently, plasmid obtained from this clone was treated with XbaI and SphI. A ca. 630 bp fragment corresponding to the downstream region of thrB was then amplified from W3110 genomic DNA by PCR using oligonucleotides AAATCTAGAATGAAACTCTACAATCTG (SEQ ID NO: 29) and AAAGCATGCGTTTAACCCTAGCGCCAC (SEQ ID NO: 30). This fragment was treated with XbaI and SphI and ligated with aforementioned treated pTH18cs1 carrying the ca. 700 bp fragment. E. coli DH5 alpha was transformed with this ligation mixture and a clone carrying pTH18cs1 including the ca. 700 bp and the ca. 630 bp fragment was obtained after selection on an LB chloramphenicol plate (10 micro g/mL) at 30° C.

The plasmid obtained from this clone was designated pTH18cs1_thrB_FR. E. coli W3110 was transformed with pTH18cs1_thrB_FR and a positive clone carrying the plasmid was obtained from an LB chloramphenicol plate (10 micro g/mL) after incubation at 30° C. Subsequently, this E. coli clone was cultured in LB chloramphenicol (10 micro g/mL) medium at 30° C. for 3 h to overnight, diluted appropriately with LB medium and incubated on an LB chloramphenicol plate (10 micro g/mL) at 42° C. overnight. The genomic DNA was extracted from colonies from this plate and used as a PCR template to verify integration of the complete plasmid pTH18cs1_thrB_FR into the E. coli genome using oligonucleotides AAAGAGCTCACCATCAGTTGCGTTATG (SEQ ID NO: 27) and AAAGCATGCGTTTAACCCTAGCGCCAC (SEQ ID NO: 30). A clone that gave rise to a ca. 5000 bp DNA fragment was selected and inoculated to a 100 mL baffled flask containing 20 mL of LB medium without chloramphenicol and was cultured at 30° C. for 4 h. This culture solution was suitably diluted in LB medium and incubated on an LB plate without chloramphenicol at 42° C. 96 colonies of the grown culture were randomly selected and grown on an LB plate without chloramphenicol and an LB chloramphenicol (10 micro g/mL) plate. Chloramphenicol sensitive colonies were selected and genomic DNA extracted from these clones was used for a PCR reaction with oligonucleotides AAAGAGCTCACCATCAGTTGCGTTATG (SEQ ID NO: 27) and AAAGCATGCGTTTAACCCTAGCGCCAC (SEQ ID NO: 30). This reaction gave rise to a ca. 1300 bp fragment corresponding to the absence of the thrB ORF and the obtained clone was designated W3110_thrB⁻.

### Example 7

### Complementation of W3110_thrB⁻

W3110 thrB⁻ as described in Example 6 was transformed with pBRGapP as described in Example 1 to obtain W3110 thrB⁻ pBRGapP, with pBRGapP_alaA_kbl_tdh as described in Example 2 to obtain W3110 thrB⁻ pBRGapP_alaA_kbl_tdh and with pBRGapP_alaA_mhpF_ltaE as described in Example 3 to obtain W3110 thrB⁻ pBRGapP_alaA_mhpF_ltaE.

These three strains and W3110 were cultured on solid medium as described in Table 1.

TABLE 1
Solid Medium Composition for Complementation of W3110_thrB⁻

| | Concentration |
|---|---|
| Component | [g/L] |
| Glucose | 40 |
| (NH₄)₂SO₄ | 10 |
| KH₂PO₄ | 1 |
| MgSO₄ 7 H₂O | 0.5 |
| FeSO₄ 7 H₂O | 0.02 |
| MnSO₄ 5 H₂O | 0.02 |
| Thiamine HCl | 0.0002 |
| Yeast Nitrogen Base Without Amino Acids | 1 |
| L-Isoleucine | 0.05 |
| Glycine | 1 |
| Agar | 15 |

Medium pH was adjusted to pH 7 with NaOH.

After 5 days of incubation at 30° C. W3110, W3110 thrB⁻ pBRGapP_alaA_kbl_tdh and W3110 thrB⁻ pBRGapP_alaA_mhpF_ltaE had formed visible colonies, while W3110 thrB⁻ pBRGapP had not.

Similarly, W3110 thrB⁻ was transformed with pBRgapP_BMD_{—}3035_kbl_tdh as described in Example 2 to obtain W3110 thrB⁻ pBRgapP_BMD_{—}3035_kbl_tdh. Single colonies of W3110 thrB⁻ pBRGapP and W3110 thrB⁻ pBRgapP_BMD_{—}3035_kbl_tdh were inoculated into 3 mL of liquid growth medium as described in Table 2 in 14 mL round bottom culture tubes. The two cultures were incubated at 30° C. and 250 rpm in an orbital shaker for 6 days. After the incubation period, the optical density of the samples at 600 nm was determined to be 0.01 AU for W3110 thrB⁻ pBRGapP and 0.31 AU for W3110 thrB⁻ pBRgapP_BMD_{—}3035_kbl_tdh with the typical appearance of bacterial growth only being present in the latter.

TABLE 2
Liquid Medium Composition for Complementation of W3110_thrB⁻

| | Concentration |
|---|---|
| Component | [g/L] |
| Glucose | 10 |
| (NH₄)₂SO₄ | 10 |
| KH₂PO₄ | 1 |
| MgSO₄ 7 H₂O | 0.5 |
| FeSO₄ 7 H₂O | 0.02 |
| MnSO₄ 5 H₂O | 0.02 |
| Thiamine HCl | 0.0002 |
| Yeast Nitrogen Base Without Amino Acids | 1 |
| L-Isoleucine | 1 |
| Glyoxylic Acid | 10 |
| Ampicillin | 0.0001 |

Medium pH was adjusted to pH 7 with NaOH.

### Example 8

### Preparation of L-Threonine from Glycine

W3110 was transformed with pBRgapP_rhtC and pBRgapP_alaA_kbl_tdh_rhtC as described in Example 4 to yield W3110 pBRgapP_rhtC and W3110 pBRgapP_alaA_kbl_tdh_rhtC, respectively. Both strains were inoculated in 3 mL LB medium, supplemented with 100 micro g/mL ampicillin in a 14 mL round bottom culture tube and grown for 20 h at 31.5° C. and 270 rpm in an orbital shaker. Subsequently, 200 micro L of this pre culture was used to inoculate 20 mL of the medium listed in Table 3. The main culture was then carried out in 150 mL baffled Erlenmeyer flasks at 31.5° C. and 270 rpm on an orbital shaker.

TABLE 3

| | Concentration |
|---|---|
| Component | [g/L] |
| Glucose | 40 |
| (NH₄)₂SO₄ | 10 |
| KH₂PO₄ | 1 |
| MgSO₄ 7 H₂O | 0.5 |
| FeSO₄ 7 H₂O | 0.02 |
| MnSO₄ 5 H₂O | 0.02 |
| Thiamine HCl | 0.0002 |
| Yeast Nitrogen Base Without Amino Acids | 1 |
| L-Isoleucine | 0.05 |
| Glycine | 1 |
| CaCO₃ | 20 |

Medium pH was adjusted to pH 7 with NaOH.

After 72 h of incubation, samples were taken and analyzed. For optical density measurements, the samples were first diluted 1:1 with 2 N HCl and then further diluted with H₂O to give an OD 660 reading of 0.3 AU or below.

HPLC analysis of glucose and organic acids was carried out on a WATERS e2695 Separations Module equipped with 2489 UV/visible Detector, 2414 Refractive Index Detector, column oven and a PC running Empower 2 software. An ULTRON PS-80H column was installed. The oven temperature was set to 60 degrees Celsius and the flow rate was 1 mL min-1 Elution was carried out isocratically using nanopure H₂O that had been adjusted to pH 2.1 with HClO₄.
L-Threonine concentration was determined on a JASCO LC-2000Plus Series HPLC system equipped with an AApak Na II-H (6.0 mm ID*80 mm L) column and a FP-2020 fluorescence detector and post-column derivatization with ortho-phthalaldehyde according to manufacturer's specification. Data acquisition was carried out on a PC equipped with JASCO ChromNAV software.
The result of the analysis is summarized in Table 4.

TABLE 4

| | | | W3110 | W3110 |
|---|---|---|---|---|
| | | | pBRgapP_rhtC | pBRgapP_alaA_kbl_tdh_rhtC |
| Consumed Glucose | | g/L | 24 | 22 |
| | | mM | 134 | 125 |
| Produced | Acetic Acid | g/L | 10 | 4.6 |
| | | mM | 166 | 76.8 |
| | | mM/mM* | 1.2 | 0.62 |
| | Lactic Acid | g/L | 0 | 0.97 |
| | | mM | 0 | 10.7 |
| | | mM/mM* | 0 | 0.09 |
| | Sum of Acids | g/L | 10.0 | 5.6 |
| | | mM | 166 | 87.6 |
| | | mM/mM* | 1.2 | 0.70 |
| | L-Threonine | g/L | 0.082 | 0.14 |
| | | mM | 0.689 | 1.18 |
| | | mM/mM* | 0.0051 | 0.0094 |
| | Cell Mass | OD 660 nm | 7.80 | 10.9 |

*mM/mM refers to molar yield of produced substance with respect to consumed glucose

As can be seen from Table 4, W3110 pBRgapP_alaA_kbl_tdh_rhtC produced 0.14 g/L of L-threonine while W3110 pBRgapP_rhtC produced 0.082 g/L. Additionally, production of organic acid byproducts was reduced unexpectedly from 10 g/L for W3110 pBRgapP_rhtC to 5.6 g/L for W3110 pBRgapP_alaA_kbl_tdh_rhtC.

### Example 9

### Preparation of L-Threonine from Glyoxylic Acid

W3110 was transformed with pBRgapP_rhtC, pBRgapP_alaA_kbl_tdh_rhtC and pBRgapP_alaA_mhpF_ltaE_rhtC as described in Example 4 to yield W3110 pBRgapP_rhtC, W3110 pBRgapP_alaA_kbl_tdh_rhtC and W3110 pBRgapP_alaA_mhpF_ltaE_rhtC, respectively. The three strains were inoculated in 3 mL LB medium, supplemented with 100 micro g/mL ampicillin in a 14 mL round bottom culture tube and grown for 20 h at 31.5° C. and 270 rpm in an orbital shaker. Subsequently, 200 micro L of this pre culture were used to inoculate 20 mL of the medium listed in Table 5, supplemented with 100 micro g/mL ampicillin. The main culture was then carried out in 150 mL baffled Erlenmeyer flasks at 31.5° C. and 270 rpm on an orbital shaker.

TABLE 5

| | Concentration |
|---|---|
| Component | [g/L] |
| Glucose | 40 |
| (NH₄)₂SO₄ | 10 |
| KH₂PO₄ | 1 |
| MgSO₄ 7 H₂O | 0.5 |
| FeSO₄ 7 H₂O | 0.02 |
| MnSO₄ 5 H₂O | 0.02 |
| Thiamine HCl | 0.0002 |
| Yeast Nitrogen Base Without Amino Acids | 1 |
| L-Isoleucine | 0.05 |
| Glyoxylic acid | 10 |
| CaCO₃ | 20 |

Medium pH was adjusted to pH 7 with NaOH.

After 48 h of incubation, samples were taken and analyzed. For optical density measurements, the samples were diluted 1:1 with 2 N HCl.

HPLC analysis of organic acids was carried out on a WATERS e2695 Separations Module equipped with 2489 UV/visible Detector, 2414 Refractive Index Detector, column oven and a PC running Empower 2 software. An ULTRON PS-80H column was installed. The oven temperature was set to 60 degrees Celsius and the flow rate was 1 mL min-1 Elution was carried out isocratically using nanopure H₂O that had been adjusted to pH 2.1 with HClO₄.

Amino acid concentrations were determined on a JASCO LC-2000Plus Series HPLC system equipped with an AApak Na II-H (6.0 mm ID*80 mm L) column and a FP-2020 fluorescence detector and post-column derivatization with ortho-phthalaldehyde according to manufacturer's specification. Data acquisition was carried out on a PC equipped with JASCO ChromNAV software.

The result of the analysis is summarized in Table 6.

TABLE 6

| | | | W3110 pBRgapP | | |
|---|---|---|---|---|---|
| | | | | alaA kbl | alaA mhpF |
| | | | rhtC | tdh rhtC | ItaE rhtC |
| Produced | Threonine | mg/L | 3.2 | 5.3 | 4.5 |
| | Acetic Acid | g/L | 1.3 | 0.8 | 0.3 |
| | Cell Mass | OD 660 nm | 0.32 | 0.28 | 0.32 |

As can be seen from Table 6, W3110 pBRgapP_alaA_kbl_tdh_rhtC produced 5.3 mg/L of L-threonine, W3110 pBRgapP_alaA_mhpF_ltaE_rhtC produced 4.5 mg/L of L-threonine while W3110 pBRgapP_rhtC produced 3.2 mg/L. Additionally, production of byproduct acetic acid was reduced unexpectedly from 1.3 g/L for W3110 pBRgapP_rhtC to 0.8 g/L for W3110 pBRgapP_alaA_kbl_tdh_rhtC and to 0.3 g/L for W3110 pBRgapP_alaA_mhpF_ltaE_rhtC.

1. An L-threonine-producing Escherichia coli comprising a pathway that produces glycine from glyoxylate with glyoxylate transaminase and a pathway selected from the group consisting of
a pathway that produces acetaldehyde from acetyl-CoA with acetaldehyde dehydrogenase, and a pathway that produces L-threonine from acetaldehyde and glycine with threonine aldolase, and
a pathway that produces 2-amino-3-oxobutyrate from glycine and acetyl-CoA with glycine C-acetyltransferase, and a pathway that produces L-threonine from 2-amino-3-oxobutyrate with threonine dehydrogenase.

2. The L-threonine-producing Escherichia coli according to claim 1, further comprising a pathway that produces glyoxylate and acetyl-CoA from malyl-CoA with malyl-CoA lyase.

3. The L-threonine-producing Escherichia coli according to claim 1, further comprising a pathway that produces malyl-CoA from malic acid and acetyl-CoA with malate thiokinase.

4. The L-threonine-producing Escherichia coli according to claim 1, further comprising an improved L-threonine export from the cell.

5. A method of producing L-threonine, the method comprising:
culturing the L-threonine-producing Escherichia coli of claim 1 in contact with a carbon source material; and
collecting L-threonine produced by the L-threonine-producing Escherichia coli.

6. A method of producing L-threonine, the method comprising:
culturing the L-threonine-producing Escherichia coli of claim 2 in contact with a carbon source material; and
collecting L-threonine produced by the L-threonine-producing Escherichia coli.

7. A method of producing L-threonine, the method comprising:
culturing the L-threonine-producing Escherichia coli of claim 3 in contact with a carbon source material; and
collecting L-threonine produced by the L-threonine-producing Escherichia coli.

8. A method of producing L-threonine, the method comprising:
culturing the L-threonine-producing Escherichia coli of claim 4 in contact with a carbon source material; and
collecting L-threonine produced by the L-threonine-producing Escherichia coli.
